(19) United States
(12) Patent Application Publication
(10) Pub. No.: US 2009/0137937 A1
(43) Pub. Date: May 28, 2009

(54) Absorbent dressing

(76) Inventors: Jens Hog Truelsen, Helsingor (DK); Brian Nielsen, Gorlose (DK)

Correspondence Address: JACOBSON HOLMAN PLLC, 400 SEVENTH STREET N.W., SUITE 600, WASHINGTON, DC, 20004, US

(21) Appl. No.: 11/991,081

(22) PCT Filed: Aug. 31, 2006
(86) PCT No.: PCT/DK2006/000467

§ 371 Date: Feb. 27, 2008

(30) Foreign Application Priority Data
Aug. 31, 2005 (DK) PA200501206
Sep. 22, 2005 (DK) 200501326
Sep. 30, 2005 (DK) 200501364

(51) Int. Cl. A61F 13/00; B32B 37/00

(52) U.S. Cl. 602/44; 156/323

(57) ABSTRACT

An absorbent wound care device comprising a layer of an absorbent material, said layer having a wound contacting surface, wherein the wound contacting surface is provided with a wound contacting layer in the form of a layer of an elastic material in the form of fibre threads forming a uniform layer of intersecting fibre threads covering at least a part of the surface of the absorbent material provides a non/low-adhesive flexible, highly absorbent wound dressing being capable of absorbing large amounts of wound exudates without giving rise to gel blocking and/or maceration, pressure marks nor leaving residues of the dressing in the wound and does not cause damage to fragile skin when removing the dressing and allows for easy access for wound exudates and slough to an absorbent layer, which ensures a fast initial absorption of an absorbing dressing.

### BACKGROUND OF THE INVENTION

1. Field of the Invention

The present invention relates to an absorbent wound care device comprising a layer of an absorbent material, said layer having a wound contacting surface, wherein the wound contacting surface is provided with a wound contacting layer and a method for preparing such a dressing.

Exudates from chronic wounds may comprise two major components, a liquid part and typically a varying amount of a more solid/highly viscous part called slough. The slough has a high content of proteins and other high-molecular compounds and may often cause problems with regard to the absorption capacity of the dressing as the slough may deposit on the surface of an absorbent element thereby causing blocking of the access to the absorbing element and preventing the uptake of the further liquid part of the exudates. This is a highly undesired situation where the absorbency of the dressing is inhibited long before the absorption capacity of a dressing has been reached.

It is desirable that wound dressings for treatment of exuding wounds are capable of absorbing exudates in such manner that both the liquid part of the exudates and the slough is able to pass the surface of the dressing freely and penetrate into the dressing thereby assuring continuous managing of the body exudates during the wound heeling process. It is advantageous for the wound healing that the slough is removed from the wound bed.

Wound dressings provided with layers for absorbing body fluids are known in the art. Absorbent layers are provided for the uptake of body fluids, especially wound exudates, so as to enable the wound dressing to keep a constant moist environment over the wound site, and at the same time avoiding maceration of the skin surrounding the wound.

Much effort has been made for enhancing the rate of uptake and increasing the capacity of absorbing body fluids of a wound dressing, in particular the absorbent layer. As absorbent layers in wound dressings have been used hydrocolloids, super absorbents, foams and synthetic materials that have extensive capacity to absorb body fluids, especially wound exudates.

However, increasing the capacity and rate of fluid uptake of a wound dressing may give rise to problems, both in assuring that the body fluids do not migrate from the wound dressing and in assuring that the wound does not dry out.

Hydrocolloid dressings are some of the most efficient and broadly used dressings, being skin-friendly, absorbent and capable of creating moist wound healing conditions. However, when used on some exuding wounds, the absorption rate tends to be too low. The advantages of the hydrocolloid dressing are the ability of creating a moist wound-healing environment and acting as a barrier against contamination with bacteria.

A problem frequently arising when treating exuding wounds is maceration. Usually the absorbent part of the dressing is optimised for absorption substantially perpendicularly to the skin, so that the skin surrounding the wound is not exposed to the exudates in order to avoid maceration of this healthy, but fragile skin. However, this limits the absorption capacity of the dressing to the part of absorbent material being directly over the wound. It is often necessary to treat the fragile skin with an ointment or barrier cream/skin conditioning paste, such as zinc paste, may be used to protect the surrounding skin for neutralising the impact of a pressure sensitive adhesive and in order to avoid the maceration.

However, the use of a paste will often inhibit both the adhesive tack of the dressing and limit the capability of absorbing exudates thus increasing the risk of leakage for products comprising relying on pressure sensitive adhesives due to adhesive failure if skin and the adhesive area may be covered with a layer of a hydrophobic material such as a zinc paste or silicone-products reducing the adherence of the adhesive, and it is time consuming for the healthcare staff to apply.

The skin surrounding leg ulcers is often very fragile and thus easily damaged. Wound dressings that allow a direct contact between pressure sensitive adhesives, such as hydrocolloid adhesives or medical acrylate based adhesives and the fragile skin surrounding the leg ulcers are often not suitable for treatment of such wounds. Traumatic removal of the adhesive dressings with damage to the fragile skin is common, and suggests the use of non-adhesive products.

Soft, flexible, absorbent and coherent dressings are in general needed for ensuring a proper treatment of exuding wounds not giving rise to pressure marks resulting from the dressing or leaving dressing residues in the wound upon removal.

Highly exuding wounds are often treated with alginate based dressings, which are capable of absorbing high amounts of exudates but require additional cover dressings. Furthermore, the risk of maceration is high due to migration of moisture in the alginate dressing along the surface of the skin. Still further, Ca-alginate fibres dissolve due to a sodium-calcium ion exchange rendering the removal of the dressing in one piece difficult.

Non-woven wound dressings are well known in the form of needle-punched non-woven materials. Most of the leading wound care suppliers have at least one needle punched dressing in their product portfolio. However, these needle-punched non-woven materials have a tendency of leaving fibre residues in the wound upon removal of the non-woven dressing.

Non-woven dressings made from pectin, Ca-alginate or CMC are traditionally used for the treatment of exuding wounds. However, these dressings have a tendency to disintegrate upon absorption due to the loss of the fibre strength. The loss of the fibre strength of the Ca-alginate fibres is due to a Ca/Na ion exchange that takes place upon absorption of the Na containing wound exudates. The CMC fibres lose the strength upon absorption due to the lack of physical bonding sites in the material. Besides loss of fibre strength in CMC dressings upon absorption, the CMC dressings tend to show film formation at the edges upon drying and stick to the wound upon removal if the wound becomes less exuding or if used on only slightly exuding wounds.

The tendency of leaving fibre residues in the wound may depend on the fibre strength and/or the dressing design.

Alginate and carboxy methylated cellulose (CMC) fibres are known to have weak fibre strengths, especially upon absorption. The weak fibre strength causes the dressings to leave residues in the wound upon removal even though the dressings are tightly needled.

Synthetic fibres, either homogeneous or bi-component, may have significant higher fibre strength and a tight needling could make fibre shedding avoidable. However, a tightly needled dressing of highly absorbable fibres may be prone to gel-blocking upon absorption of wound exudates. Therefore, the dressings may be more loosely needled in order to avoid gel blocking and hence maceration. However, for the more loosely needled non-woven dressing may be more prone to fibre shedding.

Even if the non-woven dressing should be a thermally bonded non-woven, fibre shedding may still be observed due to either low strength of the absorbent fibre of low content of the binder fibre. If a high content of binder fibre is used in order to avoid fibre shedding, stiffness and spreading of wound liquid may result.

The skin may be damaged to a degree that exudates trickle from the skin surrounding the wound, being a challenge to the function of most of the well-known dressings. Furthermore, the area of the lower limbs may pose special demands to the flexibility properties of the dressing due to the anatomical complexity of the area combined with the motility of the joints of the ankle and the malleolus.

Another important consideration in the treatment of leg ulcers is that the dressing must not give rise to formation of pressure sores. A non-adhesive dressing is usually combined with compression therapy. Thus, a dressing being soft and without sharp edges is preferred.

Leg ulcers are known to be highly exuding, and may give rise to increased risk of leakage and maceration, if the wound dressing used does not show a sufficient capacity for handling exudates with respect to time and amount. Hydrocolloid products will often be unsuitable for use in wound healing stages with medium to high level of exudates, especially if the skin is covered with lipid containing products or hydrophobic products.

2. Description of the Related Art

International Patent Application No. WO 99/61077 discloses a wound dressing or affixing tape for skin applications comprising a carrier layer that is coated on one side with an adhesive film that has skin-friendly adhesive properties, which dressing is characterized in that the carrier layer is a laminate, which comprises a polymeric film and a non-woven material and shows considerably weaker adhesive bonds to the skin than glues normally used with adhesive dressings. The adhesive layer may be perforated, especially if it includes an overlaying absorbent pad.

International Patent Application No. WO 02/03898 discloses a conformable wound dressing comprising an adhesive layer, an absorbent layer overlaying the adhesive layer and a moisture transmission layer overlying the absorbent layer, which dressing is capable of managing wound exudate. The adhesive layer may be substantially free from apertures; alternatively, it may include a plurality, preferably as a regular array, of apertures from 0.25 to 10 mm in diameter, especially from 5 to 8 mm in diameter to allow rapid uptake of exudates in the dressing. The wound dressing has a total thickness of 1.5 mm. Both Alginate and CMC fibres are described as the absorbent layer.

US Patent Application No. US 2002/0015726 discloses dressings comprising at least two components: an absorbent substrate having a first skin-facing surface and a second opposing surface; and a discontinuous coating of a semi-solid composition having an ointment-like feel overlying a portion of the first surface of said absorbent substrate. The absorbent substrate is useful as a passive dispenser of at least one active ingredient that may be contained therein. The discontinuous coating is essentially non-adherent to the skin and is useful as an active dispenser of at least one active ingredient that may be contained therein. In preferred embodiments the dressing of the invention contains at least one and more preferably at least two active ingredients intended to provide therapeutic benefit to the skin. The coating may be applied in either a regular pattern or a random pattern of elements such as straight lines, angled lines, curved lines, intersecting lines, dots, circles and geometric shapes, or in a combination of these elements, e.g. in the form of a swirling line. Ointments are not stable towards shear which may result in blocking of the dressing if the ointment is applied in large quantities which again may cause maceration of the surrounding skin.

None of these references addresses the problems being solved by the present invention and do not disclose or indicate the solution according the present invention either.

Thus, there is still a need for a non/low-adhesive flexible, highly absorbent wound dressing being capable of absorbing large amounts of wound exudates without giving rise to gel blocking and/or maceration, pressure marks nor leaving residues of the dressing in the wound and does not cause damage to fragile skin when removing the dressing and allows for easy access for wound exudates and slough to an absorbent layer, which ensures a fast initial absorption of an absorbing dressing.

One object of the present invention is to provide a wound dressing with a high permeability and a low tendency of gel blocking for treatment of highly exuding wounds and being capable of handling slough.

Another object of the present invention is to provide an absorbent wound dressing which does not shed fibres into a wound when applied to the wound and does not leave residues in the wound.

Yet another object of the present invention is to provide a dressing being soft and flexible.

Still another object of the present invention is to provide an absorbent wound care device showing only limited planar expansion, when wetted as compared to a conventional foam dressing.

A yet further object of the present invention is to provide a wound care device, which does not cause damage to fragile skin when removing the dressing.

The present invention provides a solution to the above objects enabling the use of an absorbent material comprising fibres having at least one outside portion of highly hydrophilic material and optionally binding fibres without having to rely on the presence of wicking fibres.

### SUMMARY OF THE INVENTION

The present invention relates to an absorbent wound care device comprising a layer of an absorbent material, said layer having a wound contacting surface, wherein the wound contacting surface is provided with a wound contacting layer in the form of a layer of an elastic material in the form of fibre threads forming a uniform layer of intersecting fibre threads covering at least a part of the surface of the absorbent material.

In a second aspect the invention relates to a method of preparing an absorbent wound care device comprising a layer of an absorbent material, said layer having a wound contacting surface, wherein the wound contacting surface is provided with a wound contacting layer in the form of a layer of an elastic material in the form of fibre threads forming a uniform layer of intersecting fibre threads covering at least a part of the surface of the layer, said method comprising

a) providing a layer of an absorbent material having two surfaces,
b) providing fibres of a thermoplastic material and applying a uniform layer covering at least a part of the wound contacting surface by applying the fibre treads in a predetermined pattern of intersecting fibre treads using a swirling technique, and
c) applying sufficient heat and pressure to melt together the fibre treads in the intersections forming connected (coherent) intersections and to attach the fibre treads to the layer of absorbent material.

In a third aspect the invention relates to a method of preparing an absorbent wound care device comprising a layer of an absorbent material, said layer having a wound contacting surface, wherein the wound contacting surface is provided with a wound contacting layer in the form of a layer of an elastic material in the form of fibre threads forming a uniform layer of intersecting fibre threads covering at least a part of the surface of the layer, said method comprising

a) providing a layer of an absorbent material having two surfaces,
b) providing a layer of release liner,
c) providing fibres of a thermoplastic material and applying a uniform layer covering at least a part of one surface of the release liner by applying the fibre treads in a predetermined pattern of intersecting fibre treads using a swirling technique,
d) placing the surface of the release liner being provided with the predetermined pattern of intersecting fibre treads on the wound contacting surface of the absorbent material and
e) applying sufficient heat and pressure to melt together the fibre treads in the intersections forming connected (coherent) intersections and to attach the fibre treads to the layer of absorbent material.

### BRIEF DESCRIPTION OF THE DRAWINGS

The invention is disclosed more in detail with reference to the drawings in which

FIGS. 1a-c show embodiments of the invention in cross-section,

FIG. 2 shows an embodiment of the invention seen from above, and

FIG. 3 schematically shows a side view of a further embodiment of the invention.

### DETAILED DESCRIPTION OF THE PRESENT INVENTION

The invention relates to an absorbent wound care device comprising a layer of an absorbent material, said layer having a wound contacting surface, wherein the wound contacting surface is provided with a wound contacting layer in the form of a layer of an elastic material in the form of fibre threads forming a uniform layer of intersecting fibre threads covering at least a part of the surface of the absorbent material.

In one embodiment of the invention the fibre treads are fused in the intersections forming connected (coherent) intersections and are attached to the layer of absorbent material.

It is preferred that the fibres of the uniform wound contacting layer of intersecting fibre threads is in the form of a pattern in the form of loops or other rounded figures, especially in the form of a silk loop pattern.

By applying a silk loop layer of material on the surfaces of the non-woven dressings fibre shedding can be avoided due to addition of a penetrable bonding layer on the surfaces. The silk loop layer on the first and the second surface could be of the same or different material. The silk loop layer may be in the form of fibre threads (silks) of a thermoplastic material being applied to the surface of the non-woven layer in a predetermined or random pattern.

In an embodiment of the invention the wound contacting layer is an adhesive.

Suitable adhesive materials for use according to the present invention are pressure sensitive adhesives which remain tacky after curing or setting of the composition and adhere to a variety of surfaces upon contact without the need of more than pressure from a finger or hand for application.

Suitable adhesive materials are e.g. skin friendly adhesive materials based on a block-copolymer matrix and being elastic or having a shear modulus above 1×10⁵ Pa at normal room temperature such as adhesives being bases on Kraton® elastomers based on styrene-olefin block copolymers such as a styrene-isoprene-styrene block-copolymers, oily extenders, hydrocarbon resin tackifiers and optionally hydrocolloids, e.g. an adhesive of the kind disclosed in U.S. Pat. No. 4,367,732 or EP patent application No. 1 020 195.

Furthermore hot melt adhesives may be used according to the present invention. Hot melt adhesives are only tacky upon heating and loose the tackiness after cooling to normal room temperature.

In another embodiment of the wound contacting layer is a non-adhesive material. Such a non-adhesive or hot melt material suitably has a shear modulus above 1×10⁵ Pa at normal room temperature.

The wound contacting layer is suitably made from a thermoplastic material such as a tacky or non tacky material as stated above.

In one embodiment of the invention the wound contacting layer made from a hydrophobic material. Such a material will be repelling and not stick to a wound or its surroundings and will reduce the disk of damage when removing a dressing.

In another embodiment of the invention the wound contacting layer is hydrophilic, which may assist in the transport of e.g. exudates from the wound to the absorbing material.

It has been found to be suitable when the wound contacting has area weight of between 5 g/m² and 100 g/m².

Suitable materials for use as absorbent material are e.g. non-woven materials of absorbing fibres.

According to the invention it is foreseen that a non-woven layer also may comprise non-absorbent fibres which e.g. may be binding fibres enabling a thermal bonding of the non-woven material.

Non-woven dressings are typically prepared from staple fibres (staple fibres are cut fibres of length less than from approximately 10 mm to several centimetres) which may result in leaving of fibre residues into a wound upon removal of the non-woven dressing. When a material is applied to the surface of the wound dressing in a silky loop pattern the fibres at the surface are bonded more strongly to the surface

A layer of a material applied in predetermined pattern (silky loops) can increase the hold of the fibres at the surface of a non-woven dressing such as a non-woven dressing prepared using a thermo bonding technology. The non-woven dressing can be prepared by blending a suitable amount of bi-component bonding fibres with the absorbent bi-component fibres using traditional carding techniques and then pass the carded fibres through a oven or hot calendars. Air laid fibres can also be used for this type of thermo bonded non-woven materials. The dressings of smaller sizes may be cut from a larger piece of non-woven fabric.

Binding fibres can suitably be bi-component such as bi-component PET fibres. Bi-component binding fibres do typical have a sheet/core architecture with a low melting polymer as the sheet and a high melting polymer at the core. Both the sheet and the core are typically hydrophobic. Alternatively binding fibres may be made from pure polymers having suitable melting points.

The absorbing fibres may suitable be super absorbing in order to have a high capacity for binding liquid.

Absorbing materials for use according to the present invention are suitably made from staple fibres as the invention prevents the fibre shedding problems normally observed using staple fibre-based absorbing materials.

In one embodiment of the invention the absorbing fibres are mechanically intertwined and at least a part of the absorbing fibres has a longitudinal direction forming an angle to plane of the wound contacting surface.

Mechanically intertwined fibres are suitably needle-punched fibres being simple to produce and giving many advantages.

Thus it has been found that when using needle punched absorbing materials it is possible to use of an absorbent material comprising a proportion about 50% of fibres having at least one outside portion of highly hydrophilic material without having to rely on the presence of wicking fibres for handling highly exuding wounds without risking gel blocking. The absence of wicking fibres reduces the risk of transportation of moisture laterally in the plane of the wound to the neighbouring skin and thus the risk of maceration.

A needle punched wound care device of the invention preferably has been needled to a degree ensuring that the resulting device shows only moderate expansion when wetted and also ensuring sufficient space between the swollen fibres for exudates to pass into the material and slough to be removed from the wound bed. In one embodiment of the invention, the part of the fibres forming an angle to plane of the wound contacting layer is substantially parallel.

Further it has been found that an absorbing wound care device of the invention has a sufficient cohesion to be removed in one piece essentially without leaving remnants in the wound bed and is capable of absorbing large amounts of wound exudates comprising slough without giving rise to gel blocking or physical blocking and/or maceration.

In an alternative embodiment of the invention relates to a dressing having a wound contacting surface of the absorbent non-woven staple fibre material which is corrugated. It is preferred that the absorbent non-woven staple fibre material is corrugated.

It has that above-mentioned advantages are also obtained using these embodiments.

Such embodiments has been found to have a superior performance for handling exudates comprising slough without the risking gel blocking or physical blocking.

For the purpose of the present invention the term “silk loop layer” is used to designate a layer of one or more fibre threads (silks) of a material applied to a surface of an absorbent material in the form of one or more swirling lines forming a predetermined or random pattern of the one or more threads forming a network of loops of intersecting fibre threads.

A silk loop layer is generally a porous layer having and has a porosity above approximately 50% of the total surface covered by the silk loop layer meaning that the fraction of a surface covered by the silk loop layer being available for absorption of exudates constitutes above approximately 50% of the total surface in order to ensure a proper handling of the wound exudates.

According to an embodiment of the invention the silk loop layer is in the form of a uniform layer covering at least 50% of the surface, e.g. at least 75% of the surface and especially essentially the whole surface on which the layer is applied.

A dressing according to the invention may be provided with a backing layer.

A backing layer may be of any suitable layer known per se for use as backing layer of wound dressings e.g. a foam layer, or a non-woven layer or a polyurethane, polyethylene, polyester or polyamide film, preferably a polyurethane film.

A wound care device of the invention having a skin-contacting surface being provided with a skin-friendly adhesive is optionally covered in part or fully by one or more release liners or cover films to be removed before or during application.

A protective cover or release liner may for instance be siliconized paper. It does not need to have the same contour as the device, and a number of devices may be attached to a larger sheet of protective cover. The protective cover is not present during the use of the device of the invention and is therefore not an essential part of the invention.

Furthermore, the device of the invention may comprise one or more “non touch” grip (s) known per se for applying the device to the skin without touching the adhesive layer. Such a non-touch grip is not present after application of the dressing. For larger devices it is suitable to have 2 or 3 or even 4 “non-touch” grips.

If the wound care device of the present invention is to be used as a filler for cavity wounds a silky looped pattern should be applied to both surfaces in order to avoid fibre shedding from both surfaces.

A wound contacting layer can either be applied in a predetermined pattern at a release liner which is then combined with an absorbing layer or direct at surface of an absorbing layer and may be laminated onto the surface using heat and a light pressure.

In a second aspect the invention relates to a method of preparing an absorbent wound care device comprising a layer of an absorbent material, said layer having a wound contacting surface, wherein the wound contacting surface is provided with a wound contacting layer in the form of a layer of an elastic material in the form of fibre threads forming a uniform layer of intersecting fibre threads covering at least a part of the surface of the layer, said method comprising

a) providing a layer of an absorbent material having two surfaces,
b) providing fibres of a thermoplastic material and applying a uniform layer covering at least a part of the wound contacting surface by applying the fibre treads in a predetermined pattern of intersecting fibre treads using a swirling technique, and
c) applying sufficient heat and pressure to melt together the fibre treads in the intersections forming connected (coherent) intersections and to attach the fibre treads to the layer of absorbent material.

According to the invention a silk loop layer may be applied using a swirling technique, e.g. using an application system such as a Nordson system.

Suitable materials for making silk loop layers according to the invention are thermoplastic materials. Such materials could be hydrophobic, hydrophilic or adhesive.

In one embodiment of the invention the silk loop layer is in the form of fibre treads of an adhesive material.

In another embodiment of the invention the silk loop layer is in the form of fibre treads of a material being non-adhesive.

In contrast to coating the absorbent non-woven fabric with an adhesive as described in International Patent Applications No. WO 99/61077 and WO 02/03898 the application of a silk loop layer according to the invention provides a porous layer allowing for easy access for wound exudates and slough to the absorbent fibres, which ensures a fast initial absorption. For ensuring a high initial absorption/wound contact the area weight of the applied silk loop layer should be less than 100 g/m². However, in order to avoid/reduce significantly fibre shedding the area weight should be higher than 5 g/m². Most preferably the area weight of the silk loop layer should be with in 20 g/m² to 40 g/m².

In a third aspect the invention relates to a method of preparing an absorbent wound care device comprising a layer of an absorbent material, said layer having a wound contacting surface, wherein the wound contacting surface is provided with a wound contacting layer in the form of a layer of an elastic material in the form of fibre threads forming a uniform layer of intersecting fibre threads covering at least a part of the surface of the layer, said method comprising

a) providing a layer of an absorbent material having two surfaces,
b) providing a layer of release liner,
c) providing fibres of a thermoplastic material and applying a uniform layer covering at least a part of one surface of the release liner by applying the fibre treads in a predetermined pattern of intersecting fibre treads using a swirling technique,
d) placing the surface of the release liner being provided with the predetermined pattern of intersecting fibre treads on the wound contacting surface of the absorbent material and
e) applying sufficient heat and pressure to melt together the fibre treads in the intersections forming connected (coherent) intersections and to attach the fibre treads to the layer of absorbent material.

The present invention could also be used within continence care, hygiene care and absorptions pads for hydrophilic spilling.

### DESCRIPTION OF THE PREFERRED EMBODIMENTS

The invention is now explained more in detail with reference to the drawings showing preferred embodiments of the invention.

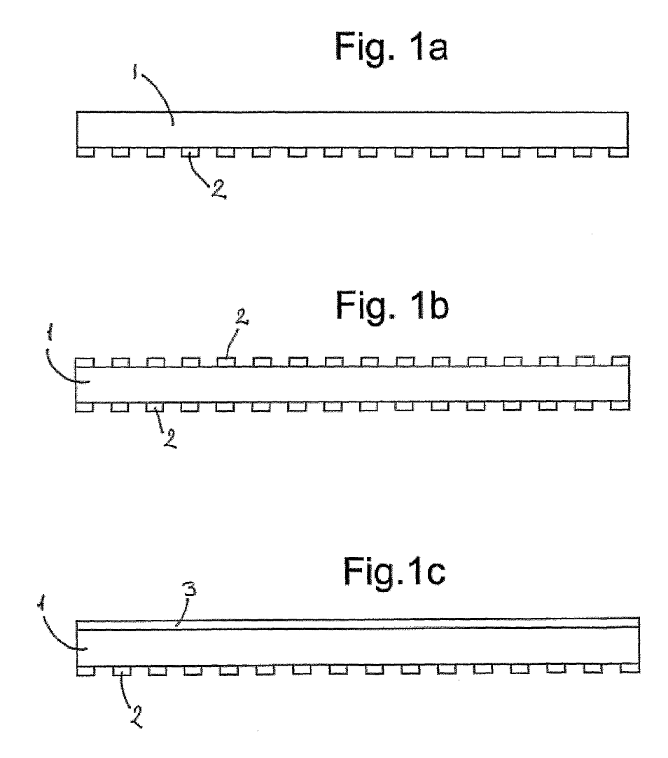

FIGS. 1a-c show embodiments of non-woven dressings according to the invention comprising a non-woven layer provided with a silk loop layer on one or more surfaces thereof. FIG. 1a shows a dressing comprising a non-woven layer (1) provided with a silk loop layer (2) on one surface, FIG. 1b shows a dressing comprising a non-woven layer (1) provided with silk loop layers (2) on two surfaces, and FIG. 1c shows a non-woven layer (1) provided with a silk loop layer (2) on one surface and a backing film (3) on the other surface.

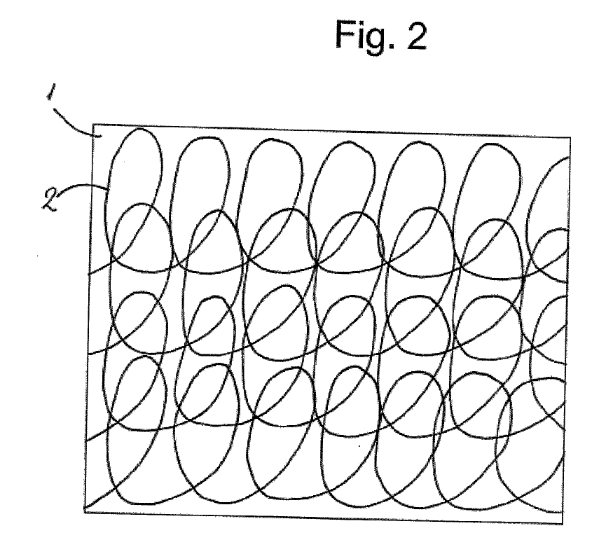

FIG. 2 shows an embodiment a dressing of the invention seen from below, showing a silk loop layer in a loop pattern.

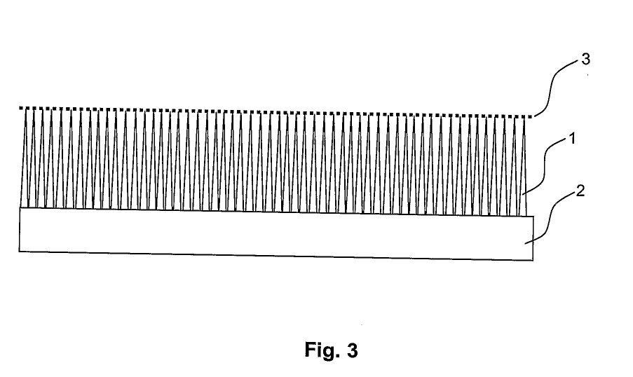

FIG. 3 shows schematically a side view of an embodiment of a non-woven dressing according to the invention comprising a non-woven layer (1) in the form of a vertically lapped material, prepared from 80% Lanseal® F fibres and 20% bi-component PET fibres. The dressing is further provided with a backing layer (3) in the form of a polyurethane film, and is further provided with a top layer (2) in the form of a silk loop layer of a block copolymer based adhesive material, which covers less than 60% of the surface.

### Materials and Methods

Lanseal® F fibres (2.9 dtex, 38 mm and 5.6 dtex, 51 mm) are obtainable from Toyobo co., Ltd. Advanced Polymer Department, 2-8, Dojima Hama 2-Chome, Kita-Ku, Osaka, 530-8230, JAPAN.

Bi-component PET fibres: Low melting PET fibres (4.4 dtex, 38-51 mm, melting temperature: 130° C.) obtainable from Huvis, 151-7 Samsung-Dong, Kangnam-gu, Seoul, Korea.

Polyurethane film: A 15 μm Polyurethane Inspire™ 1102 film obtainable from InteliCoat Technologies—UK, Wrexham Industrial Estate, Wrexham, Clwyd, LL13, 9UF, UK.

A roller-carding machine having a receive drum.

A Vulkan type stitching machine was used.

Calender, a laboratory calender having a nip of 1.3 millimetres between the rollers was used.

A complete Falubaz flat card line with feeder was used. The line was specially designed to process short staple of cotton type fibres.

A Struto experimental lapping plant (working width: 30 cm) provided with a carding section, a lapping section at which the vertical laps are formed and a thermal bonding section at which vertically lapped non-woven material is heated to a temperature above the melting temperature/glass-transition temperature (Tg) of the binding fibres.

Lamination of vertical lapped non-woven materials with a polyurethane film was carried out using a Braun flatiron type 4332.

Measurements of thickness of needle-punched non-woven materials were performed according to the standard method according to EN 29073-T2.

Measurements of thickness of lapped non-woven materials were performed using a ruler.

Absorption of Solution A (prepared according to EN13726-1) of samples was measured according Danish/European standard DS/EN 13726-1 and during application of a pressure of 40 mmHg (5332.9 Pa).

Absorption under pressure was measured by placing an oblate of the product with a predetermined diameter between a porous glass-filter placed in a Petri dish and a POM (polyoxymethylene) plate. A load equal to 40 mmHg was placed on top of the POM plate. The porous glass-filter was saturated with Solution A and the product was left to absorb for 24 h at room temperature. The residual Solution A was removed after 24 h and the load was removed. The absorbency was calculated according to following equations:

$$g \text{/} g \text{:} Absorption = \frac{{w}_{24 h} - {w}_{0 h}}{{w}_{0 h}}$$

$$g \text{/} {cm}^{2} \text{:} Absorption = \frac{{w}_{24 h} - {w}_{0 h}}{{A}_{Oblate}}$$

### Where:

w₀ₕ is the initial weight of the oblate.
w₂₄ₕ is the weight of the oblate after 24 h absorption.
A_{Oblate} is the area of the oblate.

### EXAMPLES

### Examples A1-A2

### Preparation of Needle Punched Non-Woven Materials from Lanseal® F Fibres for Use According to the Invention

Using a modernised laboratory roller carding machine made by Joseph Co. having a working width of 0.6 metres fleeces were prepared from each 0.5 kilograms of 2.9 dtex/38 mm and 5.6 dtex/51 mm Lanseal® F fibres, respectively. The fleece was collected on a drum, which served as the take-up unit, by superposing/multiplying small fleeces from the collector.

It was found empirically that 24-folded single pieces of fleece would be a suitable intermediate product for preparing a non-woven having a basis weight GSM of 150 g/m². From each 0.5 kg portion of fibres five fleece samples were prepared each having a weight of about 100 grams and a length of 1.1 metres. The fleece samples were then needled using a Vulkan type stitching machine having a working width of 1 m. The fleece was needled from both sides with 56 needle punchings on each side (112 in all). The specific weight, the thickness, the density and the absorption according to DS/EN13726-1 and under pressure of the resulting products appear from the below Table 1.

TABLE 1

| Example | | | A1 | A2 |
|---|---|---|---|---|
| Type of Lanseal F fibres | | | 2.9 dtex/38 | 5.6 dtex/51 |
| | | | mm | mm |
| Specific weight of the needle | | | 160 | 145 |
| punched non-woven, g/m² | | | | |
| Thickness, mm | | | 4 | 4 |
| Density, g/cm³ | | | 0.040 | 0.036 |
| Needle density (# per cm²) | | | 112 | 112 |
| Absorption | DS/EN | g/100 cm² | 30 | 43 |
| of Solution | 13726-1 | g/g | 27 | 23 |
| A | Under | g/100 cm² | 24 | 29 |
| | pressure | g/g | 21 | 14 |

### Examples A3-A4

### Preparation of a Needle Punched Non-Woven Materials from Lanseal® F Fibres for Use According to the Invention

In the manner described in Example 1 fleece materials were made from 160 grams and 220 grams, respectively, 5.6 dtex/51 mm Lanseal® F fibres using a roller carding machine, and the resulting materials were then needled two times using a Vulkan type stitching machine. The specific weights, the thicknesses, the densities and the absorption according to DS/EN13726-1 and under pressure of the resulting products appear from the below Table 2.

TABLE 2

| | | |
|---|---|---|
| Example | A3 | A4 |

| Specific weight of the needle | | | 140 | 190 |
|---|---|---|---|---|
| punched non-woven, g/m² | | | | |
| Thickness, mm | | | 2.7 | 3.15 |
| Density, g/cm³ | | | 0.052 | 0.06 |
| Needle density (# per cm²) | | | 178 | 178 |
| Absorption | DS/EN | g/100 cm² | 30 | 43 |
| of Solution | 13726-1 | g/g | 27 | 23 |
| A | Under | g/100 cm² | 24 | 29 |
| | pressure | g/g | 21 | 14 |

### Example A5

A part of the material from Example A4 was needled a third time on the Vulkan type stitching machine with further 56 needle punchings. The specific weight, the thickness, the density and the absorption according to DS/EN13726-1 and under pressure of the resulting product appears from the below Table 3.

TABLE 3

| Example | | | A5 |
|---|---|---|---|
| Specific weight of the needle | | | 145 |
| punched non-woven, g/m² | | | |
| Thickness, mm | | | 2.92 |
| Density, g/cm³ | | | 0.05 |
| Needle density (# per cm²) | | | 267 |
| Absorption | DS/EN | g/100 cm² | 31 |
| of Solution | 13726-1 | g/g | 23 |
| A | Under | g/100 cm² | 25 |
| | pressure | g/g | 17 |

### Examples A6-A7

Fleece materials were formed from 1000 grams 5.6 dtex/51 mm Lanseal® F fibres using a complete Falubaz flat card line with feeder. The final fleece was formed from 26 single fleeces. Such fleece was needled once on a Vulkan type stitching machine as described in Examples A1-A2. A part of the needled fleece was folded in two layers (Example A6) and another part was folded in four layers (Example A7). In both Examples the resulting products were needled twice using the Vulkan type stitching machine. The specific weights, the thicknesses, the densities and the absorption according to DS/EN13726-1 and under pressure of the resulting products appear from the below Table 4.

TABLE 4

| | | |
|---|---|---|
| Example | A6 | A7 |

| Specific weight of the needle | | | 220 | 445 |
|---|---|---|---|---|
| punched non-woven, g/m² | | | | |
| Thickness, mm | | | 5.3 | 10 |
| Density, g/cm³ | | | 0.041 | 0.045 |
| Needle density (# per cm²) | | | 267 | 267 |
| Absorption | DS/EN | g/100 cm² | 59 | 116 |
| of Solution | 13726-1 | g/g | 28 | 24 |
| A | Under | g/100 cm² | 31 | 51 |
| | pressure | g/g | 16 | 12 |

### Example A8

A sample of Approximately 1 m² of the material produced in Example A6 was calendered at 160° C. using a calender. The specific weight, the thickness, the density and the absorption according to DS/EN13726-1 and under pressure of the resulting product appears from the below Table 5.

TABLE 5

| Example | | | A8 |
|---|---|---|---|
| Specific weight of the needle | | | 207 |
| punched non-woven, g/m² | | | |
| Thickness, mm | | | 1.9 |
| Density, g/cm³ | | | 0.11 |
| Needle density (# per cm²) | | | 267 |
| Absorption | DS/EN | g/100 cm² | 47 |
| of Solution | 13726-1 | g/g | 22 |
| A | Under | g/100 cm² | 29 |
| | pressure | g/g | 12 |

### Examples A9-A13

Fleece materials were formed from each 1300 grams 2.9 dtex/38 mm Lanseal® F fibres using a flat card line. The final fleeces were formed from 26 single fleeces. Such a fleece material was needled once on a Vulkan type stitching machine as described in Example A1 (Example A9). A part of the needled fleece material was needled for a second time (Example A10) and a part thereof for a third time (Example A11) using the Vulkan type stitching machine. A sample of the needled fleece was folded to form two layers (Example A12) and another sample was folded to form four layers (Example A13) and needled once using the Vulkan type stitching machine. The specific weights, the thicknesses, the densities and the absorption according to DS/EN13726-1 and under pressure of the resulting products appear from the below Table 6.

TABLE 6

| Example | | | A9 | A10 | A11 | A12 | A13 |
|---|---|---|---|---|---|---|---|
| Specific weight of the needle | | | 144 | 120 | 115 | 180 | 400 |
| punched non-woven, g/m² | | | | | | | |
| Thickness, mm | | | 3.2 | 2.7 | 2.6 | 3.0 | 6.1 |
| Density, g/cm³ | | | 0.045 | 0.044 | 0.044 | 0.060 | 0.066 |
| Needle density (# per cm²) | | | 89 | 178 | 267 | 267 | 267 |
| Absorption | DS/EN | g/100 cm² | 40 | 38 | 27 | 38 | 68 |
| of Solution | 13726-1 | g/g | 25 | 22 | 25 | 23 | 16 |
| A | Under | g/100 cm² | 26 | 21 | 16 | 19 | 43 |
| | pressure | g/g | 17 | 18 | 16 | 12 | 9 |

### Example A14

A sample of Approximately 1 m² of the material produced in Example A10 was calendered at 160° C. using a calender. The specific weight, the thickness, the density and the absorption according to DS/EN13726-1 and under pressure of the resulting product appears from the below Table 7.

TABLE 7

| Example | | | A14 |
|---|---|---|---|
| Specific weight of the needle | | | 86 |
| punched non-woven, g/m² | | | |
| Thickness, mm | | | 1.2 |
| Density, g/cm³ | | | 0.072 |
| Needle density (# per cm²) | | | 267 |
| Absorption | DS/EN | g/100 cm² | 19 |
| of Solution | 13726-1 | g/g | 24 |
| A | Under | g/100 cm² | 13 |
| | pressure | g/g | 19 |

From the above Examples it appears that the absorption of Solution A as measured in grams solution per gram material both when measured according to DS/EN13726-1 and under pressure shows little or no dependency on the density indicating that no gel blocking occurs.

### Example B1

### Preparation of a Vertically Lapped Non-Woven Material for Use According to the Invention

Lanseal F fibres (2.9 dtex, 38 mm) from Toyobo were mixed with low melting bi-component PET fibres using the carting part on a Struto pilot plant/exhibition plant (working with: 30 cm) for making vertical lapped non-woven. The fibres were carted twice to ensure a proper mixing of the fibres. After the second carting the fleece was lapped (using a 20 mm lapping bar) and afterwards bonded by thermo bonding at 160° C. The thermo bonded vertical lapped non-woven was collected manually. By adjusting the operating speed vertical lapped non-woven having different densities as stated in the below Table 9 were prepared.

TABLE 9

| Sample | Bonding fibre | Thickness | Area density |
|---|---|---|---|
| name | (%) | (mm) | (g/m2) |
| 3 | 20 | 17 | 270 |
| 4 | 20 | 19 | 390 |
| 5 | 30 | 15 | 230 |

### Examples B2-B7

### Preparation of Vertical Lapped Non-Woven Materials for Use According to the Invention

Lanseal® F fibres (2.9 dtex, 38 mm) were mixed with low melting bi-component PET fibres using the carting part on a Struto experimental lapping plant (working width: 30 cm) for producing vertical lapped non-woven materials.

The fibres were carted twice to ensure a proper mixing of the fibres. After the second carting the resulting fleece was lapped (using a 20 mm lapping bar) and then bonded by thermo bonding at 160° C. The thermo bonded vertical lapped non-woven material was collected manually. By adjusting the weight of the fibre components vertical lapped non-woven materials having different densities were prepared using the amounts of fibres stated in the below Table 10.

TABLE 10

| | | Weight (grams) | | Overall |
|---|---|---|---|---|
| | Binding | | Lanseal ® F | Operating |
| | fibre | Binding PET | fibres | Speed |
| Example | (%, w/w) | fibres | (2.9 dtex, 38 mm) | (m/min) |
| B2 | 20 | 35 | 140 | 0.5 |
| B3 | 20 | 108 | 432 | 0.5 |
| B4 | 20 | 140 | 560 | 0.5 |
| B5 | 30 | 127 | 296 | 0.5 |
| B6 | 30 | 155 | 362 | 0.5 |
| B7 | 30 | 82 | 191 | 0.5 |

The thickness of the products of Examples B2-B7, the weight per area unit, the density and the absorption of Solution A (prepared according to EN13726-1) of the samples was measured according DS/EN 13726-1 during application of a pressure of 40 mmHg are stated in the below Table 11.

TABLE 11

| | | | | Absorption of Solution A | | | |
|---|---|---|---|---|---|---|---|
| | | | | | | Under pres- | |
| | | Weight | | (DS/EN | | sure (40 | |
| | Thick- | per area | | 13726-1) | | mmHg) | |
| | ness | unit | Density | g/100 | | g/100 | |
| Example | (mm) | (g/m²) | (g/cm³) | cm² | g/g | cm² | g/g |
| B2 | 15 | 160 | 0.011 | 68 | 26 | 37 | 17 |
| B3 | 17 | 270 | 0.016 | 98 | 24 | 41 | 13 |
| B4 | 19 | 390 | 0.021 | 112 | 24 | 57 | 12 |
| B5 | 14 | 170 | 0.012 | 62 | 33 | 34 | 16 |
| B6 | 15 | 230 | 0.015 | 78 | 24 | 28 | 17 |
| B7 | 19 | 250 | 0.013 | 77 | 33 | 37 | 18 |

As appears, the absorption of the vertically palled non-woven material in grams per gram non-woven material showed weak or no dependence on the density of the material. This indicates that no gel blocking occurs. The absorption under pressure was lower as compared to free absorption (DS/EN 13726-1). The lower absorption under pressure is believed to be ascribed partly to the reduced available free volume of the dressing and partly to reduced absorbency due to the applied pressure.

### Example B8

The vertically lapped material provided in Example B2 was combined with a 15 μm Inspire™ 1102 PU film using a Braun 4332 flatiron heated to softening of the PU film at temperature setting “2”.

### Example 1

### Application of an Adhesive Silk Loop Layer onto Non-Woven Fabrics

### Application of Silk Loop Patterned Layer of Adhesive:

Non-woven fabrics were cut into square samples of sizes 15×15 cm² for needle punched samples prepared according to Example A12 or rectangular samples of size 10×15 cm² for lapped samples prepared according to Example B3. The samples were transferred manually to a machine for application of an adhesive layer in a swirled silk loop pattern. A Kraton® based elastomer adhesive was heated to 120° C. in a melting drum and transferred through a pipeline held at 130° C. to a nozzle. In the nozzle the adhesive was heated to 200° C. for further reducing the viscosity and was then applied onto a release-liner (siliconized PET liner) in a silk loop pattern. The release-liner with the applied adhesive was then gently pressed towards the non-woven samples supported on a further release liner by passing through an oven (two heated plates with a predetermined distance being less than the height of the non-woven fabric) at 130° C. The area weight of the applied adhesive was 28 g/m².

The resulting samples showed reduced shedding of fibres as compared to corresponding samples without a silk loop adhesive layer.

1: An absorbent wound care device comprising a layer of an absorbent material, said layer having a wound contacting surface, wherein the wound contacting surface is provided with a wound contacting layer in the form of a layer of an elastic material in the form of fibre threads forming a uniform layer of intersecting fibre threads covering at least a part of the surface of the absorbent material.

2: A wound care device according to claim 1, wherein the fibre treads are fused in the intersections forming connected (coherent) intersections and are attached to the layer of absorbent material.

3: A wound care device according to claim 1, wherein the uniform layer of intersecting fibre threads is in the form of a silk loop pattern.

4: A dressing according to claim 1, wherein the wound contacting layer is an adhesive.

5: A dressing according to claim 1, wherein the wound contacting layer is non-adhesive.

6: A dressing according to claim 1 wherein the wound contacting layer comprises thermoplastic materials.

7: A dressing according to claim 1 wherein the wound contacting layer is hydrophobic.

8: A dressing according to claim 1, wherein the wound contacting layer is hydrophilic.

9: A dressing according to claim 1 wherein the wound contacting layer has area weight of between 5 g/m² and 100 g/m².

10: A dressing according to claim 1 wherein the absorbent material is in the form of a non-woven material of absorbing fibres.

11: A dressing according to claim 10, wherein the non-woven layer comprises non-absorbent fibres.

12: A dressing according to claim 10 wherein the fibres are staple fibres.

13: A dressing according to claim 10, wherein the wound contacting surface of the absorbent non-woven staple fibre material is corrugated.

14: A dressing according to claim 10 in which the absorbent non-woven staple fibre material is corrugated.

15: A dressing according to claim 10, wherein the absorbing fibres are mechanically intertwined and at least a part of the absorbing fibres has a longitudinal direction forming an angle to plane of the wound contacting surface.

16: A dressing according to claim 15 wherein the mechanically intertwined fibres are needle-punched fibres.

17: A method of preparing an absorbent wound care device comprising a layer of an absorbent material, said layer having a wound contacting surface, wherein the wound contacting surface is provided with a wound contacting layer in the form of a layer of an elastic material in the form of fibre threads forming a uniform layer of intersecting fibre threads covering at least a part of the surface of the layer, said method comprising
a) providing a layer of an absorbent material having two surfaces,
b) providing fibres of a thermoplastic material and applying a uniform layer covering at least a part of the wound contacting surface by applying the fibre treads in a predetermined pattern of intersecting fibre treads using a swirling technique, and
c) applying sufficient heat and pressure to melt together the fibre treads in the intersections forming connected (coherent) intersections and to attach the fibre treads to the layer of absorbent material.

18: A method of preparing an absorbent wound care device comprising a layer of an absorbent material, said layer having a wound contacting surface, wherein the wound contacting surface is provided with a wound contacting layer in the form of a layer of an elastic material in the form of fibre threads forming a uniform layer of intersecting fibre threads covering at least a part of the surface of the layer, said method comprising
a) providing a layer of an absorbent material having two surfaces,
b) providing a layer of release liner,
c) providing fibres of a thermoplastic material and applying a uniform layer covering at least a part of one surface of the release liner by applying the fibre treads in a predetermined pattern of intersecting fibre treads using a swirling technique,
d) placing the surface of the release liner being provided with the predetermined pattern of intersecting fibre treads on the wound contacting surface of the absorbent material and
e) applying sufficient heat and pressure to melt together the fibre treads in the intersections forming connected (coherent) intersections and to attach the fibre treads to the layer of absorbent material.
